(19) United States
(12) Patent Application Publication
(10) Pub. No.: US 2020/0121539 A1
(43) Pub. Date: Apr. 23, 2020

(54) Pediatric X-Ray And Examination Chair

(71) Applicant: Nonilon Francisco San Felipe, Mission Hills, CA (US)

(72) Inventor: Nonilon Francisco San Felipe, Mission Hills, CA (US)

(21) Appl. No.: 16/166,719

(22) Filed: Oct. 22, 2018

(51) Int. Cl. A61G 15/10; A61B 6/04

(52) U.S. Cl. CPC A61G 15/105; A61B 6/0407; A61G 2203/70; A61B 6/0478

(57) ABSTRACT

A pediatric x-ray and examination chair (PXEC) on which a person, particularly a child, can safely and comfortably be placed when an x-ray is being taken or a medical examination is performed. The PXEC has a horizontal seat platform, a rear vertical support extending upward from the back of the seat platform, a right vertical support frame and a left vertical support frame. A front side lower x-ray cartridge support and a rear upper x-ray cartridge support each maintain an x-ray cartridge adjacent a person in the PXEC while an x-ray is taken. A restraining harness with a waist belt and a front center belt secures a child within the PXEC. Located on the seat platform and within slots on each of the vertical support frames are cushions to increase the comfort of a child in the PXEC. In order to provide a calming distraction a simulated animal head, such as a pony or cat, can extend from the front of the PXEC. A speaker, along with a source playback device, facilitates sound coming from the animal head, such as neighing from the horse or meowing from the cat. The animal head with or without the sound, will calm and distract a child who is uncomfortable and/or afraid during an x-ray procedure or medical examination.

### TECHNICAL FIELD

The invention generally pertains to medical examination devices, and more particularly to a chair that is designed to safety and comfortably secure a child during an x-ray or a medical examination.

### BACKGROUND ART

All people require regular medical examinations. Often in the case of an injury, the examination must also include one or more x-rays. For young people and adults who are familiar with an examination or x-ray experience there are usually no concerns.

For children, on the other hand, an examination, and especially an x-ray can be an unfamiliar and scary experience. The most common method of reducing a child's apprehension and fear is to have the child's parent(s) present to reassure and calm the child. Another method is to divert the child's attention from what is occurring by giving the child a toy or stuffed animal. Unfortunately, in some cases, these methods do not work aid a medical professional must attempt to take an x-ray(s) or perform an examination on a screaming and crying child, which is stressful for the medical professional, child and parent(s) who are present.

The solution would be to place the child in an environment that is familiar and non-threatening. Children respond differently to various stimuli, so a solution would be to place a child in a location he/she is used to. One such location is a high chair which many children utilize.

For calming stimuli for children is an animal. While most animals are not allowed in medical facilities, a simulated animal, or even just an animal head on which the animal has a benign or smiling expression on its face can be an effective calming distraction.

A combination of a chair that resemble the experience of being in a high chair, along with an animal head that a child can touch or pet could offer a realistic solution to the problem of children being uncomfortable or scared during an x-ray or medical examination.

A search of the prior art did not disclose any literature or patents that read directly on the claims of the instant invention. However, the following U.S. patents are considered related:

| PAT. NO. | INVENTOR | ISSUED |
|---|---|---|
| 4,464,780 | Ruiz | Aug. 7, 1984 |
| 6,722,783 | Jackson, Sr. | Apr. 20, 2004 |
| 9,597,243 | Helmick, et al | Mar. 21, 2017 |

The U.S. Pat. No. 4,464,780 patent discloses a restraint apparatus for immobilizing a pediatric patient during exposure of x-ray film, particularly in connection with chest examinations. The invention permits considerable reduction in the extent of exposure area of the patient's body. Manual restraint is not required and hence non-patient exposure to radiation is avoided. Adjustment is provided for the comfortable accommodation of infants up to small children.

The U.S. Pat. No. 6,722,783 patent discloses a portable, padded chair made from x-ray transparent materials is used to comfortably support a patent while taking x-rays for medical purposes. The use of x0ray transparent materials reduces the medical assistant's and patient's risk of repeated exposure to unnecessary x-ray levels. Also, the chair allows a patient to remain in a sitting or reclining position while x-rays are taken.

The U.S. Pat. No. 9,608,243 patent discloses a medical procedure chair that includes a sitting area, a sliding seat back section, and a living hinge connecting the sitting area of the chair to a sliding seat back.

For background purposes and indicative of the art to which the invention relates, reference may be made to the following remaining patents found in the patent search.

| PAT. NO. | INVENTOR | ISSUED |
|---|---|---|
| 5,348,374 | Kuo | Sep. 20, 1994 |
| 7,452,035 | Astle, et al | Nov. 18, 2008 |
| 7,552,974 | Babikian, et al | Jun. 30, 2009 |
| 8,678,512 | Haberaecker, et al | Dec. 13, 2011 |
| 2018/0177307 | Hapler | Jun. 28, 2018 |

### DISCLOSURE OF THE INVENTION

The pediatric x-ray and examination chair (PXEC) that allows a person to be securely maintained in an upright sitting position when an x-ray is taken or a medical examination is performed. The PXEC can be used for adults, but is optimally dimensioned for a child. The reason for this is because a child is typically prone to or scared which leads to the child moving during an x-ray or examination.

The PXEC includes a horizontal seat platform, a vertical lower back support, a right vertical support frame having a slot section, a left vertical support frame having a slot section, a front side lower x-ray cartridge support and a rear upper x-ray cartridge support. Each of the two cartridge supports is utilized to hold an x-ray cartridge in a correct position adjacent an area of a child's body that is being x-rayed. The elements of the PXEC can be made of a variety of materials including plastic, metal, wood or a composite material. The PXEC can be entirely made of one of the materials, or a combination of materials can be utilized.

Various additions can be used to increase the safety and functionality of the PXEC. A cushion can be placed on the seat platform, and a cushion can be placed into the slot section on the right or left vertical support frame. A restraining harness including a waist belt and a front center belt can be used to secure a child sitting on the PXEC.

An important addition to the PXEC is a simulated animal head that is attached to the front of the seat platform and extends outward therefrom. The animal head provides a familiar and calming stimuli to a child who is uncomfortable and scared. For this purpose, a non-threatening animal is used, such as a horse, a pony, a dog, a house cat, certain wild cats, a mouse, a monkey, a giraffe or a bird. The animal head can also be made of a variety of materials including plastic, wood, a composite or a foam inner shell or solid structure that is covered by a fabric. To increase the efficacy of the animal head as a calming distraction, a speaker can be located within the animal head. The speaker allows a sound(s) that are associated with the particular animal to be heard, such as neighing of a horse or meowing from a cat. The sounds are operated by an electronic control circuit that includes a sound storage means, a playback device and an on/off switch for activating when the sound(s) is heard.

In view of the above disclosure, the primary object of the invention is to provide a pediatric x-ray and examination chair that allows a child to be securely placed and held in an upright sitting position during an x-ray or medical examination and to provide a calming distraction/environment for a child sitting on the chair.

In addition to the primary object it is also an object of the invention to provide a pediatric x-ray and examination chair that:
can be used for any child,
is easy to place a child onto and remove from,
is transportable,
can be disassembled for storage or transportation,
can be any color,
does not interfere with an x-ray or medical examination procedure,
can be used in various locations
is cost effective from both a manufacturer's and user's point of view.

These and other objects and advantages of the present invention will become apparent from the subsequent detailed description of the preferred embodiment and the appended claims taken in conjunction with the accompanying drawings.

### BRIEF DESCRIPTION OF THE DRAWINGS

FIG. 1 is an orthographic front view of a pediatric x-ray and examination chair (PXEC).

FIG. 2 is an elevational right view of the PXEC.

FIG. 3 is an elevational left view of the PXEC.

FIG. 4 is a top plan view of the PXEC.

FIG. 5 is a bottom plan view of the PXEC.

FIG. 6 is an elevational front view of the PXEC.

FIG. 7 is an elevational rear view of the PXEC.

FIG. 8 is an orthographic front view of the PXEC including a simulated animal head extending from the front.

FIG. 9 is an orthographic front view of the PXEC including a simulated animal head extending from the front, with an internal speaker in the animal head, an electronic control circuit that facilitates the operation of sound that emanates from the speaker, and an on/off switch for selecting when the control circuit is operating and the sound is heard.

### BEST MODE FOR CARRYING OUT THE INVENTION

The best mode for carrying out the invention is presented in terms that disclose a preferred embodiment of a pediatric x-ray and examination chair (PXEC 10). When taking an x-ray, or during a medical examination, children are often uncomfortable and/or scared. This results in a child refusing to remain still, crying or screaming, which makes it extremely difficult to take an x-ray or perform an examination. It is normal for a parent(s) to accompany a child for an x-ray or examination, but even the parent(s) are often unable to calm an agitated child.

This problem is exacerbated because a child is typically placed on an examination table/bed that is dimensioned to accommodate adults, with nothing holding on securing the child. One solution has been to give a child something to occupy their attention, such as a toy or a book. Unfortunately, these items are usually well-known to a child and are not new or unique enough to warrant attention and create a diversion.

The PXEC offers solution to these problems by providing a specially designed chair that is dimensioned to accommodate a child and to maintain the child in an upright position that is optimal for many x-rays and an examination. The PXEC 10 is similar to a high-chair, which most children are familiar with. In addition, the PXEC 10 includes a visual and audible distraction that offers something known and yet new at the same time.

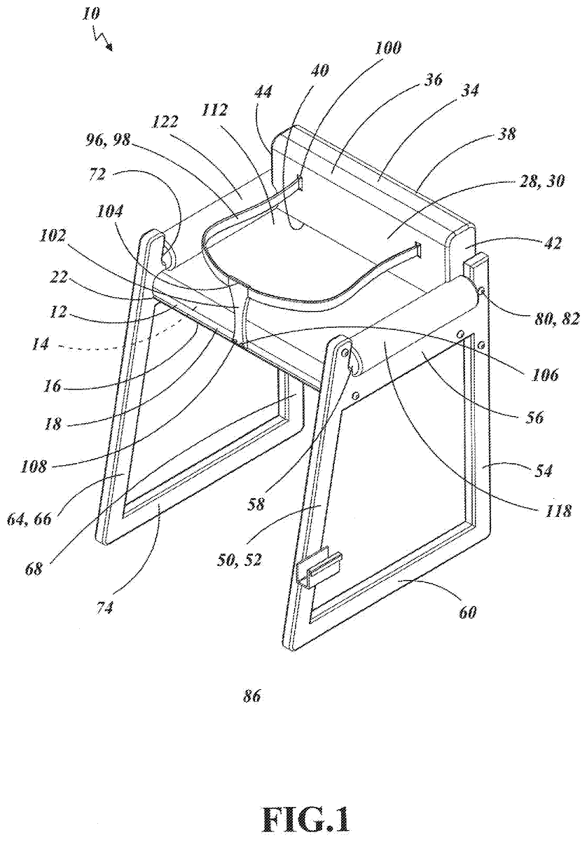

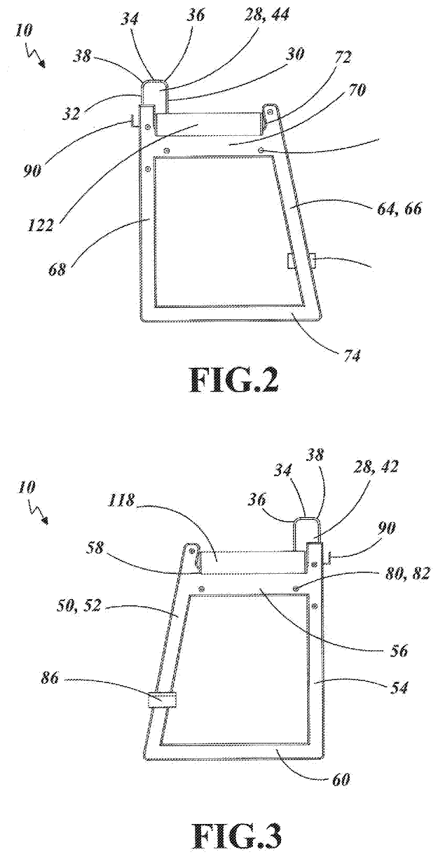

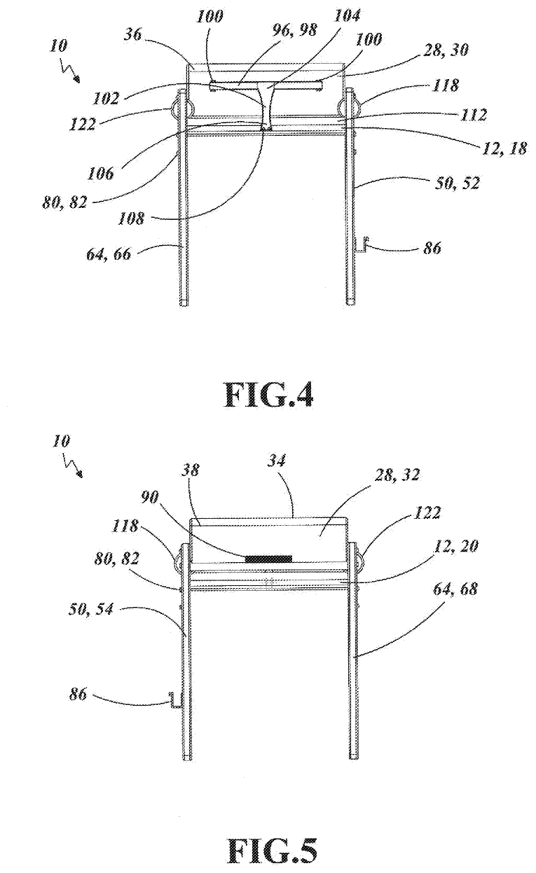

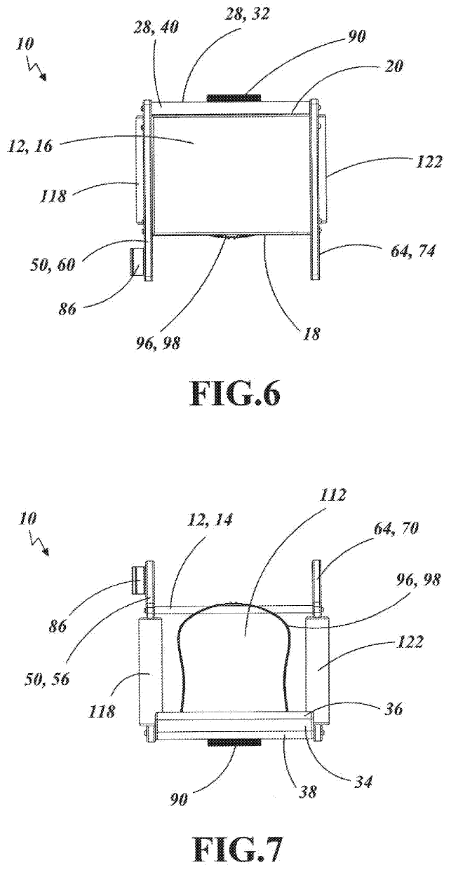

The PXEC 10, as shown in FIGS. 1-9, is comprised of the following major elements: a horizontal seat platform 12, a rear vertical support 28, a right vertical support frame 50, a left vertical support frame 64 and a restraining harness 96. To provide a calming distraction, a simulated animal head 128 is also element of the PXEC 10.

The horizontal seat platform 12 includes an upper surface 14, a lower surface 16, a front edge 18, a rear edge 20, a right edge 22 and a left edge 24.

Extending upward from the rear edge 20 of the horizontal seat platform 12 is the rear vertical support 28 that provides support for a child's lower back when the child is sitting on the horizontal seat platform. The rear vertical support 28 and maintains the child sitting in an upright straight position. The rear vertical support 28, as shown in FIGS. 1-9, is comprised of a front surface 30, a rear surface 32, an upper edge 34 preferably with an angled front corner 36 and an angled rear corner 38, a lower edge 40, a right edge 42 and a left edge 44.

Extending downward from the right edge 22 of the horizontal seat platform 12 is the right vertical support frame 50, which maintains the seat platform 12 at the desired/optimal height. The right vertical support frame 50, as shown in FIGS. 1 and 3-9, is comprised of a front member 52, a rear member 54, an upper side member 56 with an upper edge 58 having a slot section 60 extending downward, and a lower side member 62.

Extending downward from the left edge 24 of the horizontal seat platform 12 is the left vertical support frame 64, which functions in combination with the right vertical support frame 50 to maintain the seat platform 12 at the desired/optimal height. The left vertical support frame 64, as shown in FIGS. 1, 2 and 4-9, is comprised of a front member 66, a rear member 68, an upper side member 70 with an upper edge 72 that has slot section 74 extending downward, and a lower side member 76.

The two vertical support frames 50,64 are each secured to the respective right or left edges 22,24 of the horizontal seat platform 12 by attachment means 80 that are preferably comprised of a screw 82. Other attachment means including bolts and nuts or an adhesive can also be utilized. All of the members of the right and left support frames 50,64 are integrally attached.

Since the PXEC 10 is used for maintaining a child during an x-ray, a front lower x-ray cartridge support 86 is located on the front member 52 of the right vertical support frame 50, as shown in FIGS. 1-9. A rear upper x-ray cartridge support 90, is located on the rear surface 32 of the rear vertical support 28, as shown in FIGS. 1-9.

Each of the cartridge supports is U-shaped to allow a lower edge of an x-ray cartridge to be inserted into the support and maintained in position adjacent a child sitting in the PXEC 10 during an x-ray.

The restraining harness 96, as shown in FIGS. 1, 4, 6, 8 and 9, secures a child within the PXEC 10, and is preferably comprised of a waist belt 98 that extends outward from two securing openings 100 on the front surface 30 of the rear vertical support 28, and a front center belt 102 having an upper end 104 and a lower end 106, with the upper end 104 attached at the substantial center of the waist belt 98 and the lower end attached at the substantial center of the horizontal seat platform front edge 18. Both of the belts 96,102 are attached by attachment means 108 that can include screws, pins, clamps or an adhesive.

To increase the comfort of a child sitting in the PXEC 10, a platform cushion 112, as shown in FIGS. 1, 4 and 7, can be placed on the horizontal seat platform 12. Also, a right horizontal support cushion 118 can be inserted into the slot section 60 on the right vertical support frame 50, as shown in FIGS. 1 and 3-9, and a left horizontal support cushion 122 can be inserted into the slot section 74 on the left vertical support frame 64, as shown in FIGS. 1, 2 and 4-7. The two horizontal support cushions 118,122 provides a soft resilient surface on which a child can place his/her arms when sitting in the PXEC 10.

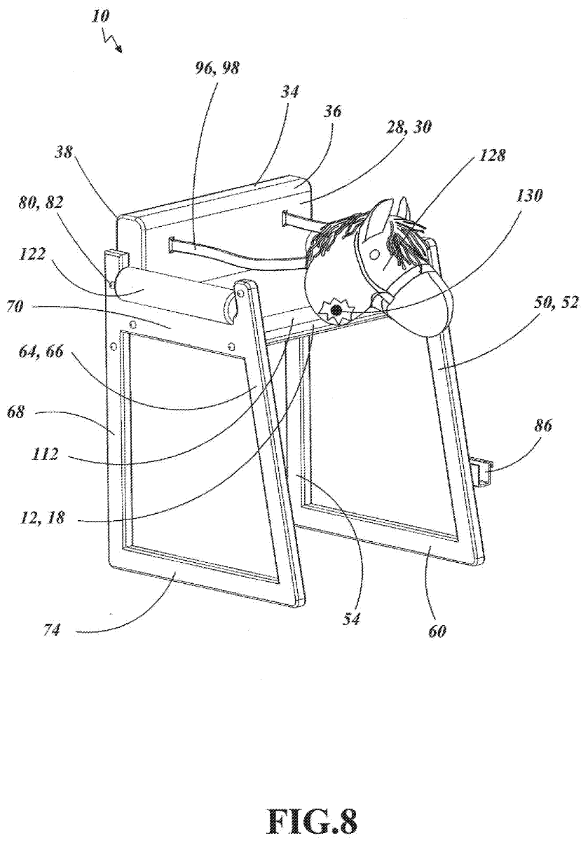

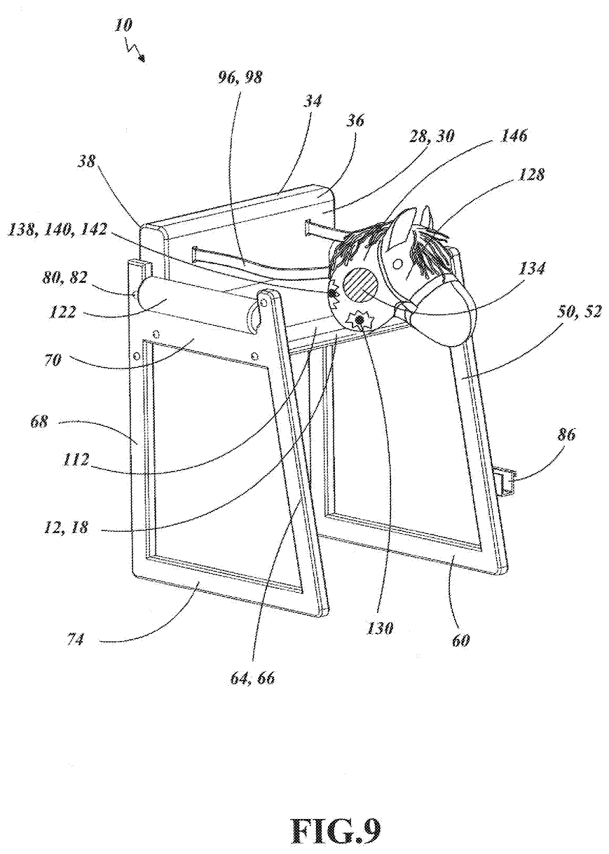

In order to provide a calming distraction, the previously disclosed simulated animal head, as shown in FIGS. 8 and 9, is utilized. The animal head 128 extends forward from and is secured by attachment means such as screws, bolts/nuts or an adhesive, to the front edge 18, of the horizontal seat platform 12, to the front edge 18 of the horizontal seat platform 12. The type of animal used for the animal head is a common non-threatening animal that most children will be familiar with, such as a horse, a pony, a dog, house cat, a wild cat, a mouse, a monkey a giraffe, or a bird. The animal head 128 can be made of a variety of materials including a fabric material that covers an integral foam structure, plastic or wood. The calming distraction is provided because children typically do not see “animals” in a medical office, and especially not as a part of a chair. A child can touch, pet, or otherwise interact with the animal head or even pretend that they are riding on the animal when in the PXEC 10.

To further increase the functionality and efficacy of the animal head 128, an internal speaker 134 can be placed within the animal head 128, as shown in FIG. 9. The speaker 134 facilitates the hearing of a sound from the animal head 128. The sound is associated with the particular animal head such as neighing from a horse or meowing from a cat. The speaker 134 functions in combination with an electronic control circuit 138, as shown in FIG. 9, that includes a sound storage means 140, a playback device 142 and a sound on/off switch 146 that controls when the sound is heard.

While the invention has been described in detail and pictorially shown in the accompanying drawings it is not to be limited to such details, since many changes and modification may be made to the invention without departing from the spirit and the scope thereof. Hence; it is described to cover any and all modifications and forms which may come within the language and scope of the claims.

1. A pediatric x-ray and examination chair (PXEC) comprising:
a horizontal seat platform comprising:
an upper surface,
a lower surface,
a front edge,
a rear edge,
a right edge, and
a left edge,
a rear vertical support extending upward from the rear edges of the horizontal seat platform and comprising:
a front surface,
a rear surface,
an upper edge with:
an angled front corner,
an angled rear corner,
a lower edge,
a right edge, and
a left edge,
a right vertical support frame secured to the right edge of the horizontal seat platform and the right edge of the rear vertical support, the right vertical support frame comprising:
a front member,
a rear member
an upper side member,
a slot section,
a lower side member, and
attachment means,
a left vertical support frame secured to the left edge of the horizontal seat platform and the left edge of the rear vertical support, the left vertical support frame comprising:
a front member,
a rear member,
an upper side ember,
a slot section,
a lower side member,
attachment means, and
a front side lower x-ray cartridge support and a rear upper x-ray cartridge support, each of the x-ray cartridge supports configured to maintain an x-ray cartridge adjacent a person in the PXEC while taking an x-ray.

2. The pediatric x-ray and examination chair of claim 1 wherein the PXEC comprises a material selected from the group consisting of wood, plastic, metal and composite.

3. The pediatric x-ray and examination chair of claim 1 wherein the right vertical support frame front member, rear member, upper side member and lower side member are integrally attached.

4. The pediatric x-ray and examination chair of claim 1 wherein the left vertical support frame front member, rear member, upper side member and lower side member are integrally attached.

5. The pediatric x-ray and examination chair of claim 1 further comprising a restraining harness configured to secure a person sitting in the PXEC, the restraining harness comprising:
a waist belt extending outward from two securing openings on the front surface of the rear vertical support, and
a front center belt with an upper end and a lower end, the upper end attached at a substantial center of the waist belt, and the lower end attached at a substantial center of the horizontal seat platform front edge.

6. The pediatric x-ray and examination chair of claim 1 further comprising a platform cushion placed on the upper surface of the horizontal seat platform.

7. The pediatric x-ray and examination chair of claim 1 further comprising a right horizontal support cushion located within the slot section of the right vertical support fume.

8. The pediatric x-ray and examination chair of claim 1 further comprising a left horizontal support cushion located within the slot section of the left vertical support frame.

9. The pediatric x-ray and examination chair of claim 1 further comprising a simulated animal head that extends outward from and is attached to the front edge of the horizontal seat platform.

10. The pediatric x-ray and examination chair of claim 9 wherein the simulated animal head further comprises an internal speaker configured to facilitate the hearing of a sound from the animal head, the sound associated with the particular animal head that is utilized, wherein an electronic control circuit includes a sound storage means, a playback device and a sound on/off switch that controls when the sound is heard.

11. The pediatric x-ray and examination chair of claim 9 wherein the simulated animal head comprises a material elected from a group consisting of fabric material that covers an internal foam structure, plastic and wood.

12. The pediatric x-ray and examination chair of claim 9 wherein the simulated animal head is selected from a group consisting of a horse, a pony, a dog, a house cat, a wild cat, a mouse, a monkey, a giraffe and a bird.

13. A pediatric x-ray and examination chair (PXEC) comprising:
a horizontal seat platform including:
an upper surface,
a lower surface,
a front edge,
a rear edge,
a right edge,
a left edge, and
a vertical lower back support extending upward from the rear edge of the horizontal seat platform and comprising:
a front surface,
a rear surface,
an upper edge with:
an angled front corner,
an angled rear corner,
a lower edge,
a right edge,
a left edge, and
a right vertical support frame secured to the right edge of the horizontal seat platform and the right edge of the rear vertical support, the right vertical support frame comprising:
a front member,
a rear member
an upper side member,
a slot section,
a lower side member,
attachment means, and
a left vertical support frame secured to the left edge of the horizontal seat platform and the left edge of the rear vertical support, the left vertical support frame comprising:
a front member,
a rear member,
an upper side member,
a slot section,
a lower side member, and
attachment means,
a front side lower x-ray cartridge support and a rear upper x-ray cartridge support, each of the x-ray cartridge supports configured to maintain an x-ray cartridge adjacent a person in the PXEC while taking an x-ray,
a restraining harness configured to secure a person sitting in the PXEC, the restraining harness comprising:
a waist belt that extends outward from two securing openings on the front surface of the rear vertical support,
a front center belt with an upper end and a lower end, the upper end attached at the substantial center of the waist belt, and the lower end attached at the substantial center of the horizontal seat platform front edge,
a platform cushion located on the upper surface of the horizontal seat platform,
a right horizontal support cushion located within the slot section of the right vertical support frame,
a left horizontal support cushion located within the slot section of the right vertical support frame, and
a simulated animal head that extends outward from and is attached to the front edge of the horizontal seat platform.

14. The pediatric x-ray and examination chair of claim 13 wherein the PXEC is dimensioned for a child.

15. The pediatric x-ray and examination chair of claim 13 wherein the PXEC comprises a material selected from a group consisting of wood, plastic, metal and composite.

16. The pediatric x-ray and examination chair of claim 13 wherein the right vertical support frame front member, rear member, upper side member, and lower side member are integrally attached.

17. The pediatric x-ray and examination chair of claim 13 wherein the left vertical support frame front member, rear member, upper side member and lower side member are integrally attached.

18. The pediatric x-ray and examination chair of claim 13 wherein the simulated animal head comprises a material elected from a group consisting of fabric material that covers an internal foam structure, plastic and wood.

19. The pediatric x-ray and examination chair of claim 13 wherein the simulated animal head is selected from a group consisting of a horse, a pony, a dog, a house cat, a wild cat, a mouse, a monkey, a giraffe and a bird.

20. The pediatric x-ray and examination chair of claim 13 wherein the animal head further comprises an internal speaker configured to facilitate the hearing of a sound that is associated with the particular animal head that is utilized, wherein the speaker is operated by an electronic control circuit that includes a sound storage means, a playback device and a sound on/off switch that controls when the sound is heard.
